(19) United States
(12) Patent Application Publication
(10) Pub. No.: US 2019/0175243 A1
(43) Pub. Date: Jun. 13, 2019

(54) MEDICAL DEVICE AND METHOD FOR THE DENERVATION OF RENAL PERIVASCULAR NERVES

(71) Applicant: CARDIOLECTRA GMBH, Berlin (DE)

(72) Inventor: Boris KEWELOH, Berlin (DE)

(21) Appl. No.: 16/277,974

(22) Filed: Feb. 15, 2019

Related U.S. Application Data
(63) Continuation of application No. PCT/DE2017/100691, filed on Aug. 16, 2017.

(30) Foreign Application Priority Data
Aug. 16, 2016 (DE) 102016115387.7

(51) Int. Cl. A61B 18/02; A61B 18/14; A61F 7/12

(52) U.S. Cl. CPC A61B 18/02; A61B 2018/00404; A61F 7/12; A61B 18/1492

(57) ABSTRACT

The present invention relates to a medical device for denervation of the renal perivascular nerves, with a catheter which comprises a flexible shaft for insertion in a patient's renal artery, wherein the shaft has an inner lumen for supplying a refrigerant and a cryoballoon arranged on the distal end of the shaft. At least one bipolar electrode is arranged on the cryoballoon to deliver an electrical pulse for stimulating the perivascular nerves. In order to guarantee ablation of the renal perivascular nerves over the entire length, the cryoballoon is embodied such that during application it touches the wall of the renal artery over a length of more than 2.5 cm in the direction of blood flow, and simultaneously ablates throughout at least 60% of its circumference and throughout a length of at least 2.5 cm.

### RELATED APPLICATIONS

This application is related to and claims priority to PCT Application No. PCT/DE2017/100691, filed Aug. 16, 2017, which claims priority to German Application No. 102016115387.7, filed Aug. 18, 2016.

### FIELD OF THE INVENTION

The present application is directed to surgical medical devices and methods of employing the same, and in particular to a device and method for the denervation of renal perivascular nerves.

### BACKGROUND OF THE PRESENT INVENTION

The invention relates to a medical device for denervation of renal perivascular nerves.

According to the related art, devices of such kind are used to treat patients with hypertension, that is to say high blood pressure.

In most cases, high blood pressure can be resolved by modifying the diet, for example by reducing salt and alcohol consumption and increasing sporting activity. Losing excess weight and avoiding stress can also lower blood pressure. Patients with elevated blood pressure may be treated with pharmaceutical preparations, which in the correct dosage and the when adjusted correctly can maintain the patient's blood pressure within acceptable limits permanently.

However, there are also patients for whom it is possible to achieve a sustained blood pressure reduction with preparations designed for long-term use.

Most of these patients suffer from a particularly pronounced hyperactivity of the sympathetic nervous system.

It has been found that notwithstanding the demonstrated multicausality of hypertension, the activity of the renal perivascular nerves is heavily implicated therein.

For this reason, particularly patients whose blood pressure cannot be adjusted to an acceptable level permanently with the aid of pharmaceutical preparations are already treated with medical devices for denervation of the renal perivascular nerves.

At the moment, denervation of the renal perivascular nerves does not always achieve the desired success.

In many patients, after a period of about 6 months following the treatment the reduction in blood pressure is so slight that the risk of an invasive procedure stands in unfavourable proportion to possible chances of recovery.

The reason for the limited efficacy of renal ablation techniques of the related art was previously unknown.

Given the above-mentioned problems, the object of the invention consists in providing a device which at least mitigates the known drawbacks of the related art and enables a sustained reduction of the blood pressure by renal denervation.

This object is solved with a medical device according to Claim 1. The other claims represent preferred embodiments of the invention.

### SUMMARY OF THE PRESENT INVENTION

In an aspect, a medical device for denervation of renal perivascular nerves includes: a catheter with a flexible shaft for insertion into a patient's renal artery; the shaft has an inner lumen for supplying a refrigerant and a cryoballoon arranged on the distal end of the shaft to receive the refrigerant; and at least one bipolar electrode arranged on the cryoballoon to deliver an electrical pulse for stimulating the perivascular nerves. The cryoballoon is arranged such that when configured as inserted into a renal artery for treatment, it is configured to touch the wall of the renal artery over a length of at least 2.5 cm, and simultaneously configured to ablate the tissue surrounding the renal artery throughout at least 60% of its circumference and throughout the length of at least 2.5 cm.

In another aspect, the medical device is characterized in that the catheter is connectable to a refrigerant source, further comprising a controller to control the refrigerant source; the controller is configured to control the refrigerant source to supply refrigerant to the cryoballoon based on a value of the patient's blood pressure.

In a further aspect, the medical device, the catheter is connected to a pressure sensor for determining the patient's blood pressure.

In yet another aspect, the medical device includes a control unit, the control unit being connected to the at least one bipolar electrode and being configured to deliver an electrical pulse for stimulating the perivascular nerve, wherein; when an increase in blood pressure by a predetermined limit value following a stimulation of the perivascular nerves by the at least one electrode is detected by the pressure sensor, the control unit one of: starts or maintains a flow of refrigerant to the cryoballoon.

In yet a further aspect, the medical device includes a control unit, the control unit being connected to the at least one electrode and configured to deliver an electrical pulse to stimulate the perivascular nerves, wherein, when an increase in blood pressure by a predetermined limit value in response to a stimulation of the perivascular nerves by the at least one electrode is not detected by the pressure sensor, the controller interrupts a flow of refrigerant to the cryoballoon.

In an aspect, the medical device includes a control unit, the control unit having a test mode, the control unit being configured to cool the cryoballoon to a temperature at which the conduction of stimuli by the renal perivascular nerves is attenuated or entirely suppressed but an ablation of the renal perivascular nerves does not take place.

In a yet further aspect, in the medical device, a measurement of a lesion depth formed by an ablation of the renal perivascular nerves is made through an impedance measurement with the aid of the electrodes and the controller, and wherein the controller is configured to terminate the ablation of the renal perivascular nerves in the event a prescribed value for the calculation of the lesion depth has been determined by means of the impedance measurement.

In another aspect, in the medical device, treatment is ended after a cumulative ablation time of 18 minutes.

In a further aspect, the medical device further includes at least 2 electrodes, the electrodes being arranged on the outside of the cryoballoon.

In an aspect, in the medical device,the catheter includes an additional cold-insulated channel to enable a flow of blood to the kidney during ablating of the tissue surrounding the renal artery.

In a further aspect, a method for treating hypertension in a patient, includes the steps of ablating the renal perivascular nerves throughout a length of at least 2.5 cm and throughout the entire circumference of the renal artery with a cryoablation balloon. The method includes using the medical device above.

In yet a further aspect, in the method for treating hypertension in a patient, a refrigerant is supplied to the cryoballoon for ablation, and wherein the supply is controlled on the basis the blood pressure increase measured during a rise of a heartbeat.

In an aspect, the method for the treatment of hypertension in a patient includes the steps of: a controller first initiating an emission of one or a sequence of pulses or a frequency via at least one electrode; then, the controller using a measured blood pressure or a correlated value checking whether an increase in blood pressure has occurred within a predefined time interval above a predetermined limit value; if such increase has occurred, then an ablation phase is initiated for a predetermined time; after an ablation phase has ended, another stimulation and possibly ablation phase is initiated after a period of between 5 and 20 minutes; if a rise in blood pressure is not detected again in this phase, or according to variant of the embodiment during a further stimulation phase, the treatment is ended, otherwise, the cycle starts from the beginning again.

In another aspect, a method for the denervation of renal perivascular nerves includes: providing a catheter having a flexible shaft for insertion into a patient's renal artery, the shaft having an inner lumen for supplying a refrigerant and a cryoballoon arranged on the distal end of the shaft to receive the refrigerant as well as providing at least one bipolar electrode arranged on the cryoballoon to deliver an electrical pulse for stimulating the perivascular nerves. The method includes the steps of: inserting the catheter into the renal artery for treatment such that the cryoballoon is in contact with the inner wall of the renal artery over a length of at least 2.5 cm; and, simultaneously ablating the tissue surrounding the renal artery throughout at least 60% of its circumference and throughout the length of at least 2.5 cm.

### BRIEF DESCRIPTION OF THE DRAWINGS

FIG. 1 shows the medical device according to the invention with refrigerant source and control unit.

FIG. 2 shows a side view of a first embodiment of the catheter.

FIG. 3 is a cross sectional representation along plane A-A of the catheter of FIG. 2.

FIG. 4 is a cross sectional representation of a second embodiment of the catheter.

FIG. 5 is a cross sectional representation of a third embodiment of the catheter.

### DETAILED DESCRIPTION OF THE EMBODIMENTS OF THE PRESENT INVENTION

The medical device for denervation of renal perivascular nerves according to the invention is equipped with a catheter having a flexible shaft for introduction into a patient's renal artery.

The shaft has an inner lumen which is designed to supply a refrigerant. At the distal end of shaft, the medical device has a cryoballoon. This cryoballoon is connected to the lumen of the shaft and is supplied with a refrigerant through the shaft. The evaporation of the refrigerant in the balloon has the effect of lowering the surface of the balloon to a temperature preferably between −30 and −80° C., with the aid of which the renal perivascular nerves are ablated.

At least one bipolar electrode is disposed on the surface of the balloon and connected to a power source for emitting an electrical pulse or a sequence of pulses or a frequency to stimulate the perivascular nerves.

This excitation of the perivascular nerves is used for a feedback loop to enable the success of the treatment to be assessed.

In this context, the medical device according to the invention is characterized in that the cryoballoon is embodied such that during application it touches the wall of the renal artery (in the direction of the blood flow) over a length of more than 2.5 cm, preferably at least 3 cm and particularly preferably at least 3.5 cm, and simultaneously ablates throughout a length of at least 2.5 cm, preferably at least 3 cm and particularly preferably at least 3.5 cm (in the direction of the blood flow).

In the expanded state, the cryoballoon has a diameter of between 3 and 7 mm, preferably between 4 and 6 mm.

Inspired by the medical principle of ablating the smallest possible volume of tissue to achieve medical success (in this context the ablation and interruption of the nervous conduction of stimuli by the renal perivascular nerves), the provision of an ablation surface this large using devices of the related art was not desired. Especially since the risk of a vasoconstriction, blood clot formation and vascular perforation must be treated critical.

Moreover, an interruption of the nervous conduction was also already achieved by ablation of certain regions of the renal artery, so that ablations of large areas were deliberately avoided.

It was therefore the more surprising that a sustained reduction of blood pressure in the patient treated can be achieved with a plurality of thorough cryoablations of the renal perivascular nerves, ideally over the entire length of the renal artery and preferably over the entire circumference thereof.

Accordingly, the geometric dimensions of the medical device according to the invention are designed for the purpose of ablating the renal artery over most of the length thereof and preferably over its entire circumference.

Furthermore, the medical device according to the invention relies on cold (cryoenergy) and not radio frequency energy (heat). Cryoablations tear the inner wall of the blood vessel less often, blood clots form less frequently and the danger of perforating the vessel is reduced.

During application, the catheter on which the cryoballoon is disposed is connected to a refrigerant source and a controller for the refrigerant source.

According to a preferred embodiment, in such case the refrigerant source and/or the supply of the refrigerant to the cryoballoon is controlled on the basis of a blood pressure value of the patient during the treatment.

According to a further embodiment, the catheter is equipped with a pressure sensor. This enables a direct (invasive) blood pressure measurement to be carried out during the denervation. The blood pressure value is captured and forwarded to the controller, which controls the refrigerant source and/or the supply of the refrigerant to the cryoballoon as a function of this value.

But it is also possible, and provided according to a further variant of the invention, that the controller is connected to a blood pressure cuff, and the current blood pressure is correspondingly measured indirectly. However, in this case as well the refrigerant source and/or the supply of the refrigerant to the cryoballoon is controlled on the basis of the measured blood pressure.

However, an invasive blood pressure measurement offers still more advantages: Preferably, the rate of pressure increase, that is to say the delta mmHg/s is measured. This takes place particularly preferably during the rise of a heartbeat, as a measure of cardiac sufficiency and of blood pressure. In this way, it is possible to diagnose much sooner and more confidently whether the renal perivascular nerves have already been sufficiently ablated or if they can still be stimulated.

According to a further variant of the invention, the power source is connected to the controller via at least one electrode for the purpose of emitting an electrical pulse or an electrical frequency. The controller then first initiates the emission of one or a sequence of pulses or a frequency via at least one electrode. This process may also be referred to as the stimulation phase.

Then, the controller uses the measured blood pressures or a correlated value to check whether an increase in blood pressure has occurred within a predefined time interval and above a predetermined limit value.

If this is the case, an ablation phase is then initiated by actuating the refrigerant source or an ablation phase is continued or also repeated for a predetermined time.

In this context, the blood pressure or a correlated value which was measured shortly before or during the stimulation of the perivascular nerves with the aid of electrical pulses is compared with the value blood pressure value preferably after a period of between 15 seconds and 5 minutes, more preferably after a period of between 2 and 4 minutes after the start of the stimulation phase.

The perivascular nerves are stimulated e.g. with a frequency of 20 Hz, a voltage of 15 Volt (amplitude) and for a duration of at least 10 ms.

The limit value of the minimal pressure increase, at which an ablation phase is initiated or maintained, is preferably 5 mmHG.

The ablation phase preferably lasts for between 2 and 3 minutes, wherein the balloon surfaces have preferably reached a temperature below −50° C.

According to a preferred embodiment, the ablation phase is ended when the blood pressure does not rise above a value that was measured before the start of the stimulation phase, despite stimulation which also takes place during the ablation phase.

After an ablation phase has ended, the controller is preferably designed to start another stimulation and possibly ablation phase after a period of between 5 and 20 minutes.

The controller starts another stimulation phase particularly preferably between 5 and 10 minutes after the end of an ablation phase. However, if an increase in blood pressure by a predetermined limit value is not captured upon stimulation, the controller starts another stimulation phase after a further 5 to 10 minutes. If a rise in blood pressure is not detected again in this phase, or according to variant of the embodiment during a further stimulation phase, the treatment is ended. Otherwise, the cycle starts from the beginning again.

According to a further embodiment, is provided that the electrodes take impedance measurements. According to a preferred embodiment the impedance measurement is used by the controller to calculate a value for the lesion depth. It is preferably further provided that a limit value for the lesion depth is stored in the controller. Accordingly, the treatment cycle is terminated if the impedance measurement conducted with the aid of the electrodes returns a value indicating that a permissible lesion depth associated therewith has been exceeded.

According to a further embodiment, the cryoballoon is designed in such manner that it has a preferably cold-insulated channel extending in the lengthwise direction on the outside, which channel extends radially preferably over between 10 and 40% of the circumference thereof and more preferably over between 25 and 35% of the circumference thereof. The channel preferably has the shape of a cylindrical sector.

Through this canal, blood can flow to the kidney during the ablation, which lasts 2-3 minutes. This prevents any damage to the renal tissue due to repeated ischaemia.

According to one embodiment, the cryoballoon has a trip of material which is not stretchable or very poorly stretchable material at the location of the channel which is to be formed. This strip, which may be attached to the catheter or also to the cryoballoon by adhesion or which partially replaces the original stretchable material of the cryoballoon, prevents the cryoballoon from expanding at this point, ensuring that a channel remains unblocked in the cross section of the artery, so that blood flow to the kidney is maintained during the treatment as well.

If the cylindrical sector spans a circumferential range of more than 180°, it can be assured that the entire circumference of the vessel all comes into contact with the cryoballoon when it is applied twice, if during the treatment the cryoballoon is rotated at least once about the longitudinal axis by a minimum angle that is larger than the angle spanned by the cylindrical sector of channel 8.

According to another embodiment, it is provided that whereas the cryoballoon is cylindrical, there is a bypass channel—in other words a tunnel—through the balloon, which enables a flow of blood to the kidney during the treatment as well.

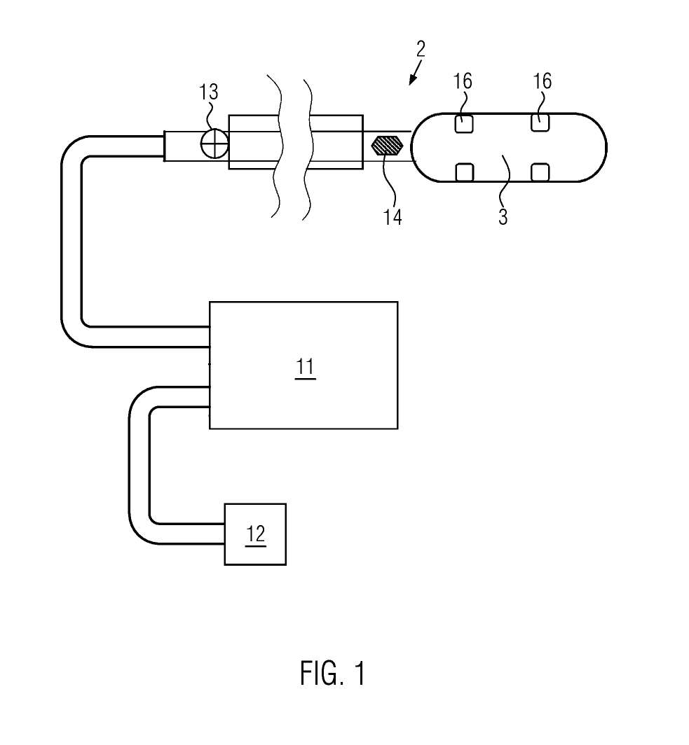

FIG. 1 shows the medical device according to the invention 1. The device comprises a catheter 2 with a flexible contacting surface which is designed to be inserted into a human artery and in particular the renal artery.

The catheter 2 at the distal end is designed to be expandable and during application it forms a cryoballoon 3 for ablating body tissue.

An apparatus for measuring blood pressure 14 is arranged on the shaft of the catheter. A marking 13 is created close to the proximal end of the catheter 2, which marking is used as a reference when the catheter is rotated about its longitudinal axis during the treatment.

An electrical connection and a connection for a refrigerant are provided at the proximal end of the catheter, which connect the catheter 2 to a controller 11. The controller itself is also connected to a source for a refrigerant 12 and during application controls the supply of refrigerant to the cryoballoon 3 arranged at the distal end of the catheter 2.

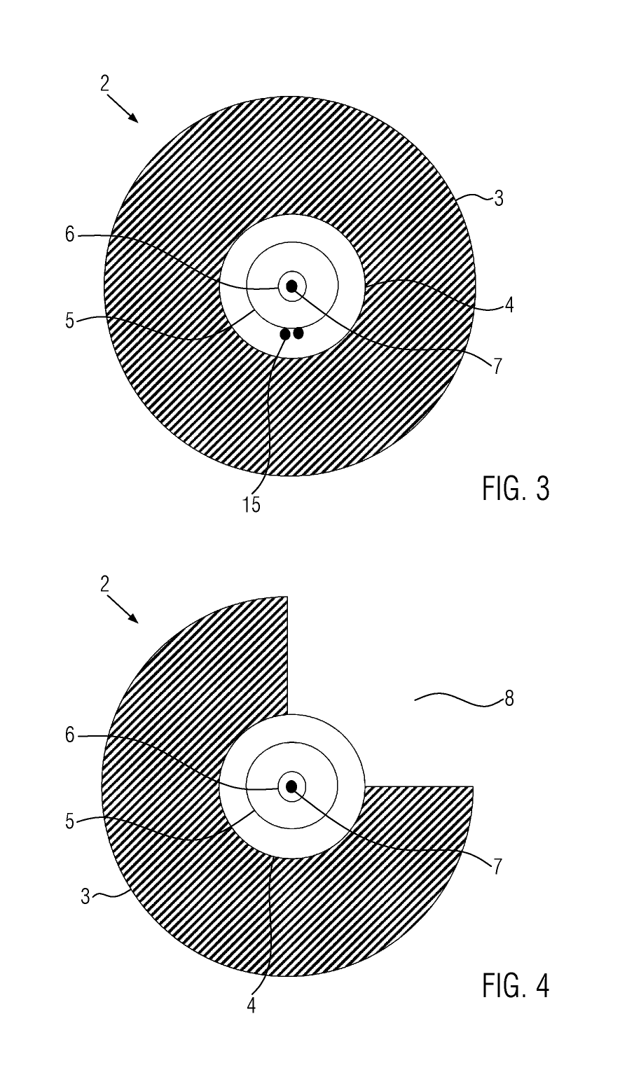

The controller 11 is also connected to the pressure sensor 14 via electrical lines 15 (see FIG. 3) for determining the blood pressure. Further electrical lines connect four electrodes 16 arranged on the cryoballoon 3 to the controller 11.

During application, at least one of the electrodes 16 stimulates the renal perivascular nerves of a patient. The controller 11 detects whether a blood pressure increased or the increase of a value correlated to the blood pressure has taken place within a previously defined time interval and above a prescribed limit value in response to the stimulation by the electrode(s) 16.

If this is the case, the controller 11 introduces refrigerant from the refrigerant source 12 into the cryoballoon 3 through the catheter 2 and initiates an ablation of the renal perivascular nerves. A marking 13 is provided on the catheter with which it is possible to fix a reference point for the rotation of the catheter about its longitudinal axis and to check whether a desired minimum rotation has been completed during the treatment.

This is particularly advantageous in embodiments in which the cryoballoon 3 is designed such that its cross section does not touch the entire circumference of the arterial vessel wall during a treatment procedure.

In this case, it is essential for the treatment that the ablation of the renal perivascular nerves be carried out in at least two and exactly two treatment steps. During the treatment, the catheter must then be rotated at least once through a prescribed minimum angle about the longitudinal axis of the catheter 2.

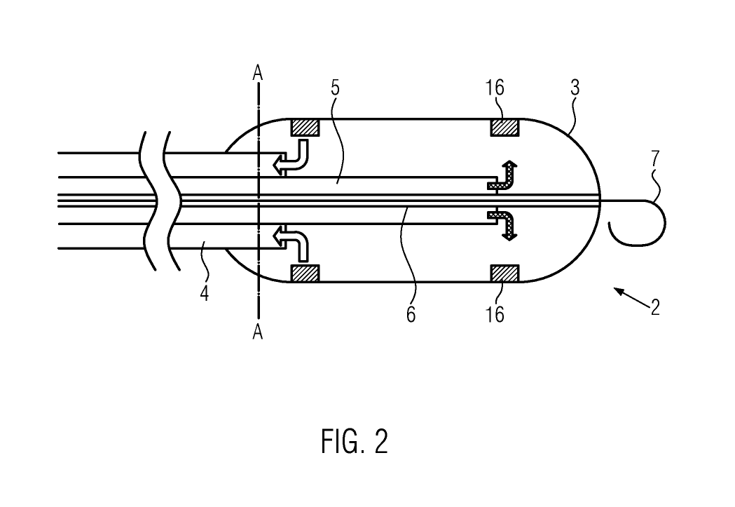

FIG. 2 shows a longitudinal section through the catheter in a first embodiment which has a symmetrical cross section. The catheter has 3 lumens. The innermost lumen 6 is designed to accommodate a J-wire and is dimensioned accordingly. During treatment, the J-wire helps when introducing the catheter into a patient's renal artery.

As soon as the catheter 2 has reached it intended position in the patient's renal artery, compressed air is fed into the cryoballoon 3 through the outermost lumen 4, causing it to expand and make contact with the vessel wall of the artery. A stimulation of the renal perivascular nerves is then carried out by means of the electrodes 16, which normally results in an increase in blood pressure.

If an increase in blood pressure is determined, liquid refrigerant (preferably N₂O) passes into the cryoballoon 3 through the distal end of the lumen 5 and evaporates. This evaporation generates cold which is transferred to the tissue via the wall of the cryoballoon 3 and causes denervation due to ablation. The expanded gases are then passed through the outermost lumen 4 to the proximal end of the catheter 2 and discharged to the anaesthetic gas scavenging system of the catheter laboratory/operating room.

In a variant of this embodiment, it may also be provided that the liquid refrigerant enters the cryoballoon 3 through holes which are arranged at intervals in the wall of the catheter 2 over the length of the cryoballoon.

FIG. 3 shows a cross section of the embodiment of the catheter represented in FIG. 2 along section line A-A. Outwards from the inside, FIG. 3 shows the J-wire 7, the lumen 6 for guiding the J-wire, the lumen 5 for guiding the liquid refrigerant, the lumen 4 for initial filling of the cryoballoon 3 with compressed air and for subsequent removal of the gas-phase refrigerant.

In this embodiment, the electrical lines 15 which are connected to the pressure sensor 14 and the electrodes 16 are accommodated in the lumen for removing the gas-phase refrigerant (N₂O).

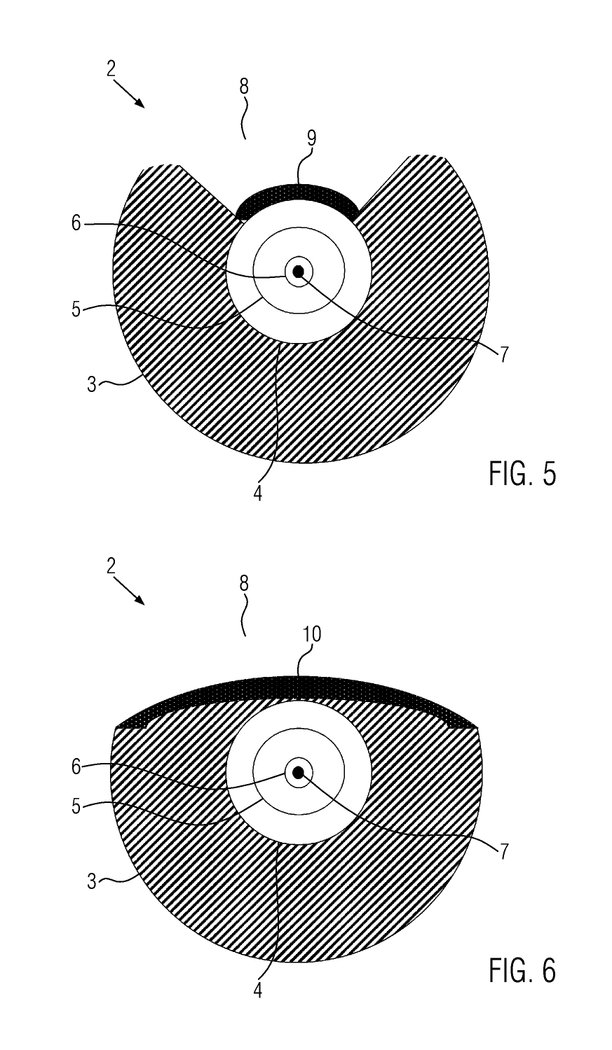

FIG. 4 represents a second embodiment of the catheter 2 and particularly of the cryoballoon 3. In order to minimise the risk of undersupply to the kidney and consequently of renal infarction during the treatment because of blood congestion in front of the cryoballoon 3, this embodiment of the cryoballoon 3 is not symmetrical, but rather corresponds to the shape of a cylindrical sector. In the embodiment represented here, a channel 8 is left free by the cryoballoon 3, through which channel blood flows towards the kidney even during a treatment procedure, thereby supplying it with the essential oxygen.

In this embodiment the cryoballoon 3 has the shape of a cylindrical sector, wherein the circumference of the cryoballoon spans a circumferential range of more than 180°, preferably more than 230°. If the cylindrical sector has a circumferential range greater than 180°, it can be guaranteed that the entire circumference of the vessel wall comes into contact with the cryoballoon 3 if the cryoballoon 3 is rotated at least once through a minimum angle greater than the angle spanned by the cut out cylindrical sector of channel 8 about its longitudinal axis during the treatment.

The catheter 2 preferably has one or more marking(s) 13 at a proximal end, which serve to enable a determination to be made as to whether the rotation about the longitudinal axis performed during the treatment is greater than the angle spanned by the channel 8. In this way, it may be ensured that the entire circumference of the renal artery is touched by the cryoballoon 3 during the treatment and the renal perivascular nerves are consequently ablated over the entire length and circumference of the artery.

FIG. 5 is a further embodiment, in which the cryoballoon 3 does not touch entire inner wall of the renal artery during treatment and keeps a channel 8 free for the blood. For this purpose, a strip 9 of a poorly elastic material is attached, for example glued to the catheter 2 before and after the cryoballoon 3. This strip 9 prevents the cryoballoon 3 from expanding in the region of the strip 9.

According to a preferred embodiment, the strip 9 is made from a material with low thermal conductivity, or an insulating material is provided in addition to the strip 9 and prevents the blood flowing through the channel 8 from freezing or clumping during treatment.

FIG. 6 is a further embodiment in which the cryoballoon 3 does not touch entire inner wall of the renal artery during treatment. In this case, a layer 10 which stretches little or not at all is provided and is attached to the surface of the cryoballoon 3, or the stretchable or folded surface of the cryoballoon 3 is replaced with a layer that stretches little or not at all and/or does not unfold. In this manner too, a channel 8 can be kept free and the supply of blood to the kidney is never completely blocking during the treatment by the cryoballoon 3, so that the risk of a renal infarction is reduced significantly. As with the embodiment represented in FIG. 5, in this variant as well it may be provided that the layer 10 is made from a material having low thermal conductivity.

1. A medical device for denervation of renal perivascular nerves, comprising:
a catheter comprising a flexible shaft for insertion into a patient's renal artery, the shaft having an inner lumen for supplying a refrigerant and a cryoballoon arranged on the distal end of the shaft to receive the refrigerant;
at least one bipolar electrode arranged on the cryoballoon to deliver an electrical pulse for stimulating the perivascular nerves;
characterized in that the cryoballoon is arranged such that when configured as inserted into a renal artery for treatment, it is configured to touch the wall of the renal artery over a length of at least 2.5 cm, and simultaneously configured to ablate the tissue surrounding the renal artery throughout at least 60% of its circumference and throughout the length of at least 2.5 cm.

2. The medical device according to claim 1, characterized in that the catheter is connectable to a refrigerant source, further comprising a controller to control the refrigerant source, wherein the controller is configured to control the refrigerant source to supply refrigerant to the cryoballoon based on a value of the patient's blood pressure.

3. The medical device according to claim 2, wherein the catheter is connected to a pressure sensor for determining the patient's blood pressure.

4. The medical device according to claim 2, further comprising a control unit, the control unit being connected to the at least one bipolar electrode and being configured to deliver an electrical pulse for stimulating the perivascular nerve, wherein; when an increase in blood pressure by a predetermined limit value following a stimulation of the perivascular nerves by the at least one electrode is detected by the pressure sensor, the control unit one of: starts or maintains a flow of refrigerant to the cryoballoon.

5. The medical device according to claim 2, further comprising a control unit, the control unit being connected to the at least one electrode and configured to deliver an electrical pulse to stimulate the perivascular nerves, wherein, when an increase in blood pressure by a predetermined limit value in response to a stimulation of the perivascular nerves by the at least one electrode is not detected by the pressure sensor, the controller interrupts a flow of refrigerant to the cryoballoon.

6. The medical device according to claim 2, further comprising a control unit, the control unit having a test mode, the control unit being configured to cool the cryoballoon to a temperature at which the conduction of stimuli by the renal perivascular nerves is attenuated or entirely suppressed but an ablation of the renal perivascular nerves does not take place.

7. The medical device according to claim 2, wherein a measurement of a lesion depth formed by an ablation of the renal perivascular nerves is made through an impedance measurement with the aid of the electrodes and the controller, and wherein the controller is configured to terminate the ablation of the renal perivascular nerves in the event a prescribed value for the calculation of the lesion depth has been determined by means of the impedance measurement.

8. The medical device according to claim 2, wherein treatment is ended after a cumulative ablation time of 18 minutes.

9. The medical device according to claim 1, further comprising at least 2 electrodes, the electrodes being arranged on the outside of the cryoballoon.

10. The medical device according to claim 2, wherein the catheter comprises an additional cold-insulated channel to enable a flow of blood to the kidney during ablating of the tissue surrounding the renal artery.

11. Method for treating hypertension in a patient, comprising the steps of:
ablating the renal perivascular nerves throughout a length of at least 2.5 cm and throughout the entire circumference of the renal artery with a cryoablation balloon.

12. The method for treating hypertension in a patient according to claim 11 using the medical device according to claim 1.

13. The method for treating hypertension in a patient according to claim 11, whereby a refrigerant is supplied to the cryoballoon for ablation, and wherein the supply is controlled on the basis the blood pressure increase measured during a rise of a heartbeat.

14. The method for the treatment of hypertension in a patient according to claim 11, comprising the steps of:
a controller first initiating an emission of one or a sequence of pulses or a frequency via at least one electrode,
then, the controller using a measured blood pressure or a correlated value checking whether an increase in blood pressure has occurred within a predefined time interval above a predetermined limit value,
if such increase has occurred, then an ablation phase is initiated for a predetermined time,
after an ablation phase has ended, another stimulation and possibly ablation phase is initiated after a period of between 5 and 20 minutes,
if a rise in blood pressure is not detected again in this phase, or according to variant of the embodiment during a further stimulation phase, the treatment is ended, otherwise, the cycle starts from the beginning again.

15. A method for the denervation of renal perivascular nerves comprising:
providing a catheter having a flexible shaft for insertion into a patient's renal artery, the shaft having an inner lumen for supplying a refrigerant and a cryoballoon arranged on the distal end of the shaft to receive the refrigerant; and,
providing at least one bipolar electrode arranged on the cryoballoon to deliver an electrical pulse for stimulating the perivascular nerves;
the method comprising the steps of:
inserting the catheter into the renal artery for treatment such that the cryoballoon is in contact with the inner wall of the renal artery over a length of at least 2.5 cm; and,
simultaneously ablating the tissue surrounding the renal artery throughout at least 60% of its circumference and throughout the length of at least 2.5 cm.
